SEQUENCE LISTING (10 sequences)

SEQ ID NO: 1 - DNA, 201 bp, Ovis aries
  source 1..201: mol_type = genomic DNA; note = Sheep SNP molecular marker
ctgcagccccgcgttatgccaggcggccaagactgcagggatgcgaagccgggccgcagcgaggctatgagagtcgccttgggagccggtgcctcatgacytctgcccgggacctggggaaagaattgaggcctttagtattaagctccaggctttggatagcattgtgttctccacttcccatttccaaccctgggacct

SEQ ID NO: 2 - DNA, 20 bp, synthetic construct
  source 1..20: mol_type = other DNA; note = Forward primer for detecting the genotype of SNP molecular marker
ggagccggtgcctcatgact

SEQ ID NO: 3 - DNA, 19 bp, synthetic construct
  source 1..19: mol_type = other DNA; note = Forward primer for detecting the genotype of SNP molecular marker
gagccggtgcctcatgacc

SEQ ID NO: 4 - DNA, 25 bp, synthetic construct
  source 1..25: mol_type = other DNA; note = Reverse primer for detecting the genotype of SNP molecular marker/Primer R
caatgctatccaaagcctggagctt

SEQ ID NO: 5 - DNA, 41 bp, synthetic construct
  source 1..41: mol_type = other DNA; note = Primer X
gaaggtgaccaagttcatgctggagccggtgcctcatgact

SEQ ID NO: 6 - DNA, 40 bp, synthetic construct
  source 1..40: mol_type = other DNA; note = Primer Y
gaaggtcggagtcaacggattgagccggtgcctcatgacc

SEQ ID NO: 7 - DNA, 21 bp, synthetic construct
  source 1..21: mol_type = other DNA; note = fluorescent probe A
gaaggtgaccaagttcatgct

SEQ ID NO: 8 - DNA, 21 bp, synthetic construct
  source 1..21: mol_type = other DNA; note = fluorescent probe B
gaaggtcggagtcaacggatt

SEQ ID NO: 9 - DNA, 21 bp, synthetic construct
  source 1..21: mol_type = other DNA; note = quenching probe A
agcatgaacttggtcaccttc

SEQ ID NO: 10 - DNA, 21 bp, synthetic construct
  source 1..21: mol_type = other DNA; note = quenching probe B
aatccgttgactccgaccttc

(19) United States
(12) Patent Application Publication
(10) Pub. No.: US 2025/0122583 A1
(43) Pub. Date: Apr. 17, 2025

(54) SHEEP SINGLE NUCLEOTIDE POLYMORPHISM (SNP) MOLECULAR MARKER AND USE THEREOF IN DETECTING RESISTANCE TO BRUCELLOSIS IN SHEEP

(71) Applicant: Institute of Animal Sciences of CAAS, Beijing (CN)

(72) Inventors: Xiaohong HE, Beijing (CN); Lin JIANG, Beijing (CN); Shangjie WU, Beijing (CN); Yuehui MA, Beijing (CN)

(21) Appl. No.: 18/910,384

(22) Filed: Oct. 9, 2024

(30) Foreign Application Priority Data
Oct. 11, 2023 (CN) 202311311458.4

(51) Int. Cl. C12Q 1/6888; C12Q 1/6844; G16B 20/20

(52) U.S. Cl. CPC C12Q 1/6888; C12Q 1/6844; G16B 20/20; C12Q 2600/156

(57) ABSTRACT

A sheep single nucleotide polymorphism (SNP) molecular marker and use thereof in detecting resistance to brucellosis in sheep are provided, relating to the technical field of molecular marker-assisted breeding. A sheep SNP molecular marker is provided, where the sheep SNP molecular marker is associated with resistance to brucellosis in sheep, and has a nucleotide sequence shown in SEQ ID NO: 1 in which a T/C polymorphism is located at 101^{st} bp. The sheep SNP molecular marker can accurately detect a resistance level of sheep to the brucellosis, and is used for the selection of brucellosis-resistant sheep as well as molecular marker-assisted breeding of the resistance to brucellosis in sheep. The sheep SNP molecular marker effectively improves a breeding efficiency of sheep with brucellosis resistance.

### CROSS REFERENCE TO RELATED APPLICATION

This patent application claims the benefit and priority of Chinese Patent Application No. 202311311458.4, filed with the China National Intellectual Property Administration on Oct. 11, 2023, the disclosure of which is incorporated by reference herein in its entirety as part of the present application.

### REFERENCE TO SEQUENCE LISTING

A computer readable XML file entitled “GWP20240906409_seqlist”, that was created on Oct. 9, 2024, with a file size of about 11,949 bytes, contains the sequence listing for this application, has been filed with this application, and is hereby incorporated by reference in its entirety.

### TECHNICAL FIELD

The present disclosure relates to the technical field of molecular marker-assisted breeding, and in particular relates to a sheep single nucleotide polymorphism (SNP) molecular marker and use thereof in detecting resistance to brucellosis in sheep.

### BACKGROUND

Single nucleotide polymorphism (SNP) markers have been widely used as genetic markers in the field of genetic breeding. Compared with traditional breeding methods, molecular marker-assisted breeding greatly improves breeding efficiency and saves breeding time. Sheep are important livestock that provide human with products such as meat and fur. Brucellosis is a zoonosis caused by infection with pathogens from Brucella. Clinical symptoms of the brucellosis include undulant fever, arthritis, and reproductive disorders (such as orchitis and epididymitis in males, as well as abortion and infertility in females). Brucella has the characteristics of immune evasion and latent infection, among which Brucella ovis is the most virulent and infectious, not only causing huge economic losses to the sheep farming industry, but also becoming a major safety hazard to the public health. Molecular markers related to the resistance to brucellosis in sheep, a host animal, are discovered and applied to assist in disease-resistant breeding of the sheep. This can fundamentally cut off the transmission pathway of brucellosis, and reduce environmental pollution and drug residue hazards caused by the abuse of antibiotics. Therefore, it is necessary to explore a molecular marker related to resistance to brucellosis in sheep.

### SUMMARY

The present disclosure provides a sheep SNP molecular marker and use thereof in detecting resistance to brucellosis in sheep.

In the present disclosure, a genome-wide association study (GWAS) is conducted on different sheep breeds to find an SNP molecular marker in a sheep genome that is associated with an resistance to brucellosis. Sheep population verification with a large sample size has proved that the SNP molecular marker is significantly correlated with the resistance to brucellosis in sheep and can be used to detect a resistance level of sheep to the brucellosis. To facilitate detection, the present disclosure further develops a primer combination for detecting the SNP molecular marker.

Based on the above findings, the present disclosure provides the following technical solutions:

In a first aspect, the present disclosure provides a sheep SNP molecular marker, where the sheep SNP molecular marker is associated with resistance to brucellosis in sheep, and has a nucleotide sequence shown in SEQ ID NO: 1 in which a polymorphism is T/C at 101^{st} base pair (bp).

In the present disclosure, the sheep SNP molecular marker has a polymorphic site located at position 35,704,228 of chromosome 3 of a sheep reference genome with a version of Oar_v1.0 on November 2017, and the polymorphism is the T/C.

The sheep SNP molecular marker shows significant correlation with sheep resistance to brucellosis, and can accurately identify the resistance of different sheep to the brucellosis. The sheep SNP molecular marker is used for molecular marker-assisted breeding and breed improvement of resistance to brucellosis in sheep, thereby improving an efficiency of molecular breeding.

Based on a polymorphic site at position 35,704,228 of chromosome 3 on a sheep reference genome on November 2017 with the version of Oar_v1.0. To facilitate detection, the present disclosure develops primers for amplifying the SNP molecular marker for upstream and downstream sequences of the polymorphic site. By combining the upstream and downstream sequences of the polymorphic site in the SNP, the sequence shown in SEQ ID NO: 1 is obtained in the present disclosure. It can be understood by those skilled in the art that sequence fragments of different lengths can be developed based on the SNP site and their upstream and downstream sequences for amplification and detection of the SNP site. Therefore, the sequence shown in SEQ ID NO: 1 does not constitute a limitation on the SNP molecular marker disclosed in the present disclosure. As long as the sequence fragments containing the polymorphic site at position 35,704,228 of chromosome 3 on the sheep reference genome with the version of Oar_v1.0 on November 2017 are included, they are within the protection scope of the SNP molecular marker disclosed in the present disclosure.

In some examples, the sheep SNP molecular marker has a nucleotide sequence shown in SEQ ID NO: 1, the polymorphic site is located at 101^{st} bp of the nucleotide sequence shown in SEQ ID NO: 1, and the polymorphism is the T/C.

SEQ ID NO: 1:

CTGCAGCCCCGCGTTATGCCAGGCGGCCAAGACTGCAGGGATGCGAAGCCG GGCCGCAGCGAGGCTATGAGAGTCGCCTTGGGAGCCGGTGCCTCATGACYTCTGCC CGGGACCTGGGGAAAGAATTGAGGCCTTTAGTATTAAGCTCCAGGCTTTGGATAGC ATTGTGTTCTCCACTTCCCATTTCCAACCCTGGGACCT. Y is the polymorphic site of the SNP molecular marker, and Y is selected from the group consisting of T and C.

In some examples, the sheep SNP molecular marker is prepared by amplifying a sheep genome as a template using primers having sequences shown in SEQ ID NO: 2 to SEQ ID NO: 4.

In some examples, a genotype being selected from the group consisting of TT and CC corresponds to high resistance to the brucellosis while the genotype being TC corresponds to low resistance to the brucellosis in the polymorphic site of the sheep SNP molecular marker.

Among the SNP molecular marker, sheep with a “TT” homozygous genotype and a “CC” homozygous genotype have significantly higher resistance to brucellosis than sheep with a “TC” heterozygous genotype.

In a second aspect, the present disclosure provides a primer combination for amplifying the sheep SNP molecular marker.

According to the genomic location of the polymorphic site of the SNP molecular marker provided above and its upstream and downstream sequences, those skilled in the art can develop various types of primers for amplifying the SNP molecular marker.

The above primers can be any primers that can be used to detect the genotype of SNP molecular marker.

Preferably, the primer combination includes primers having sequences shown in SEQ ID NO: 2 to SEQ ID NO: 4, where the primers having sequences shown in SEQ ID NO: 2 to SEQ ID NO: 3 serve as forward primers, and the primer having a sequence shown in SEQ ID NO: 4 serves as a reverse universal primer.

SEQ ID NO: 2:
5′-GGAGCCGGTGCCTCATGACT-3′;
SEQ ID NO: 3:
5′-GAGCCGGTGCCTCATGACC-3′;
SEQ ID NO: 4:
5′-CAATGCTATCCAAAGCCTGGAGCTT-3′.

The primer combination can achieve efficient amplification and genotyping for the SNP molecular marker.

Competitive allele-specific PCR, or KASP, is a technology that can conduct high-precision bi-allelic typing of SNPs through specific fluorescent probes. The KASP has the advantages of stable and accurate analysis, low cost, and high efficiency, and is easy to achieve high throughput and automation. Therefore, in order to achieve efficient detection, the present disclosure develops a KASP primer combination for detecting the SNP molecular marker based on the KASP.

Preferably, the KASP primer combination includes a first forward primer, a second forward primer, and a reverse universal primer; where the first forward primer includes a specific fluorescent tag sequence and a sequence shown in SEQ ID NO: 2 that are ligated in sequence, and the second forward primer includes a specific fluorescent tag sequence and a sequence shown in SEQ ID NO: 3 that are ligated in sequence; the reverse universal primer has a nucleotide sequence shown in SEQ ID NO: 4.

The fluorescent tags of the first forward primer and the second forward primer are different.

In some examples, the primer combination includes forward primers having sequences shown in SEQ ID NO: 5 to SEQ ID NO: 6 and a reverse universal primer having a sequence shown in SEQ ID NO: 4.

In a third aspect, the present disclosure provides a kit, including the primer combination.

To facilitate detection, the kit may further include other reagents for PCR amplification, including but not limited to DNA polymerase, PCR reaction buffer, probe, dNTP, Mg²⁺, and water.

The above reagents can be packaged separately or provided as a premix after mixing.

The kit can be used in any one of the following items:
1) detecting or assisting in detecting a resistance level of sheep to brucellosis;
2) screening or identifying a sheep individual with high resistance to the brucellosis;
3) early prediction of resistance to brucellosis in sheep;
4) molecular marker-assisted breeding for sheep with resistance to the brucellosis; and
5) breed improvement for the sheep with the resistance to the brucellosis.

In a fourth aspect, the present disclosure provides use of a sheep SNP molecular marker or a detection primer of the sheep SNP molecular marker in any one of the following 1) to 8):
1) use in detecting or assisting in detecting a resistance level of sheep to brucellosis;
2) use in preparation of a reagent for detecting or assisting in detecting the resistance level of the sheep to the brucellosis;
3) use in screening or identifying a sheep individual with high resistance to the brucellosis;
4) use in preparation of a reagent for screening or identifying the sheep individual with the high resistance to the brucellosis;
5) use in early prediction of resistance to brucellosis in sheep;
6) use in preparation of a reagent for the early prediction of the resistance to brucellosis in sheep;
7) use in molecular marker-assisted breeding for sheep with resistance to the brucellosis; and
8) use in breed improvement for the sheep with the resistance to the brucellosis; where

the sheep SNP molecular marker has a polymorphic site located at position 35,704,228 of chromosome 3 of a sheep reference genome with the version of Oar_v1.0, and a polymorphism is T/C.

In some examples, in the use, the sheep SNP molecular marker has a nucleotide sequence shown in SEQ ID NO: 1 in which a polymorphism is T/C at 101^{st} bp.

In some examples, in the use, the sheep SNP molecular marker has a nucleotide sequence shown in SEQ ID NO: 1, the polymorphic site is located at 101^{st} bp of the nucleotide sequence shown in SEQ ID NO: 1, and the polymorphism is the T/C.

In some examples, in the use, a detection primer of the sheep SNP molecular marker includes primers having sequences shown in SEQ ID NO: 2 to SEQ ID NO: 4, where the primers having sequences shown in SEQ ID NO: 2 to SEQ ID NO: 3 serve as forward primers, and the primer having a sequence shown in SEQ ID NO: 4 serves as a reverse universal primer.

In the use, a genotype being selected from a sheep population consisting of TT and CC corresponds to high resistance to the brucellosis while the genotype being TC corresponds to low resistance to the brucellosis in the polymorphic site of the sheep SNP molecular marker.

In the use, sheep with the genotypes “TT” and “CC” of the SNP molecular marker are selected as a parent to allow breeding for resistance to brucellosis.

In a fifth aspect, the present disclosure provides a method for detecting resistance of sheep to brucellosis, including: detecting a genotype of an SNP molecular marker in a sheep genome; where the sheep SNP molecular marker has a polymorphic site located at position 35,704,228 of chromosome 3 of a sheep reference genome with the version of Oar_v1.0 on November 2017, and the polymorphism is the T/C.

A genotype being selected from the group consisting of TT and CC corresponds to high resistance to the brucellosis while the genotype being TC corresponds to low resistance to the brucellosis in the polymorphic site of the sheep SNP molecular marker.

In some examples, the sheep SNP molecular marker has a nucleotide sequence shown in SEQ ID NO: 1 in which a polymorphism is T/C at 101^{st} bp.

In some examples, the sheep SNP molecular marker has a nucleotide sequence shown in SEQ ID NO: 1, the polymorphic site is located at 101^{st} bp of the nucleotide sequence shown in SEQ ID NO: 1, and the polymorphism is the T/C.

Preferably, the method includes the following steps:
1) extracting a genomic DNA from a sheep individual to be tested;
2) conducting PCR amplification using the genomic DNA from step 1) as a template and using primers having sequences shown in SEQ ID NO: 2 to SEQ ID NO: 4; and
3) analyzing a genotype of the SNP molecular marker in an obtained PCR amplification product, and determining a resistance level of the sheep individual to brucellosis according to the genotype; where a genotype being selected from the sheep consisting of TT and CC corresponds to high resistance to the brucellosis while the genotype being TC corresponds to low resistance to the brucellosis in the polymorphic site of the sheep SNP molecular marker.

The beneficial effects of the present disclosure include at least: a sheep SNP molecular marker is provided, where the sheep SNP molecular marker is significantly correlated with resistance to brucellosis in sheep and can more accurately detect the resistance of sheep to brucellosis. The sheep SNP molecular marker can realize the early prediction of resistance to brucellosis in sheep, is not limited by the age and gender of the sheep, and can even be accurately conducted when the sheep is just born. The sheep SNP molecular marker can be used for the selection of sheep with anti-brucellosis and molecular marker-assisted breeding of sheep with resistance to brucellosis, and can significantly promote the breeding of sheep with anti-brucellosis and effectively improve a breeding efficiency. Accordingly, the sheep SNP molecular marker is of great significance for the development and utilization of the excellent economic characteristics of sheep breeds and the protection and rational use of breed resources.

### BRIEF DESCRIPTION OF THE DRAWINGS

To describe the technical solutions in the examples of the present disclosure or in the prior art more clearly, the following briefly describes the accompanying drawings required for describing the examples or the prior art. Apparently, the accompanying drawings in the following description show some examples of the present disclosure, and a person skilled in the art may still derive other drawings from these accompanying drawings without creative efforts.

FIG. 1 shows genotyping results of the polymorphic site of the SNP molecular marker in Example 2 of the present disclosure; and

FIG. 2 shows expanded population verification results in a sheep population of the SNP molecular marker in Example 2 of the present disclosure.

### DETAILED DESCRIPTION OF THE EMBODIMENTS

To make the objectives, technical solutions, and advantages of the present disclosure clearer, the following clearly and completely describes the technical solutions in the present disclosure with reference to the accompanying drawings in the present disclosure. Apparently, the described examples are some but not all of the examples of the present disclosure. All other examples obtained by those of ordinary skill in the art based on the examples of the present disclosure without creative efforts shall fall within the protection scope of the present disclosure.

The present disclosure provides a SNP molecular marker associated with resistance to brucellosis in sheep, where a polymorphic site of the SNP molecular marker is located at 35,704,228 bp of chromosome 3 of sheep. There is a T/C base mutation at the polymorphic site, which has a significant correlation with the resistance to brucellosis in sheep. It is speculated that the mutation of the site may affect the sheep's ability to clear Brucella. At the polymorphic site, compared with a “TC” heterozygous genotype, sheep with a “TT” homozygous genotype and a “CC” homozygous genotype show stronger resistance to brucellosis. The polymorphic site of the SNP molecular marker is based on a sheep genome sequence with an information version of Oar_v1.0, November 2017. The SNP molecular marker has important guiding significance for distinguishing and screening sheep with resistance to brucellosis through genotypes, and can improve the accuracy and efficiency in screening of resistance to brucellosis in sheep.

In the present disclosure, there is no particular limitation on a method for detecting a genotype of the SNP molecular marker, and conventional genotype detection methods in the art may be used. In a specific example, KASP is conducted to detect the genotype of the SNP molecular marker in the genome of a sheep individual to be tested.

In the present disclosure, the SNP molecular marker or a detection primer thereof related to the resistance to brucellosis in sheep provided can be used in combination with other SNP molecular markers or detection primers thereof related to the resistance to brucellosis in sheep to allow identification of the resistance to brucellosis in sheep.

### Example 1 Development of SNP Molecular Marker Associated with Resistance to Brucellosis in Sheep

In this example, sheep populations of 4 breeds, namely Suffolk, East Frisian, White Suffolk, and Texel, were used as samples to develop an SNP molecular marker associated with the resistance to brucellosis in sheep through GWAS, as follows:

The Suffolk, East Friesian, White Suffolk, and Texel sheep were raised under the same conditions and naturally infected with brucellosis. Blood samples were collected from sheep, and competitive enzyme-linked immunosorbent assay (cELISA), indirect enzyme-linked immunosorbent assay (iELISA), and fluorescence polarization assay (FPA) were conducted to detect whether the sheep were infected with brucellosis. The positive determination threshold lines of each method were: cELISA: 30, iELISA: 15, FPA: 20, and those above the threshold lines were determined as positive. If the test results of the above three methods were all positive, it was determined to be infected with Brucella; if the results were all negative, it was determined to be healthy; the rest results were determined to be suspicious. Finally, 25 sheep diagnosed with brucellosis were selected as an experimental group, and 25 sheep diagnosed as healthy were selected as a control group. GWAS was conducted on the above 50 sheep.

The blood DNA samples of the above 50 sheep were subjected to whole genome resequencing using a BGI T7 sequencing platform, with a sequencing depth of 20× and a total sequencing volume of 2.6 T. The sequencing data were aligned and quality controlled. The fastp software was used for data filtering, and the GTX software was used for alignment and variation detection, and an aligned VCF file with a size of 71.08 GB was obtained. The quality control software PLINK was used with the following criteria: --mind 0.1 --geno 0.1 --maf 0.05 --hwe 1e-5, and a number of effective SNPs after quality control was 24,683,444. The principal component analysis was conducted using the software PLINK, code: plink --bfile filename --pca 10 --out filename_pca --chr-set 27 --allow-extra-chr. Quantitative traits (iELISA values) were selected and GWAS was conducted using GEMMA (Version 0.95) software, with MLM(y=γCov+Xβ+Zα+Wμ+e) as a model and Genome assembly Oar_rambouillet_v1.0 as a reference genome. SNP annotation and enrichment analysis were conducted, where a tool used for SNP site annotation was https://asia.ensembl.org/index.html, the reference genome was Genome assembly Oar_rambouillet_v1.0; a tool used for GO enrichment analysis was https://biit.cs.ut.ee/gprofiler/gost, the reference genome was Homo sapiens (Human), Ovis aries (Sheep); and a tool used for KEGG enrichment analysis was: https://david.ncifcrf.gov, the reference genome was Homo sapiens (Human), Ovis aries (Sheep).

In the GWAS, the −log₁₀(P) values of the Top 1,000 SNPs ranged from 4.56 to 7.36, and 100 genes were annotated. Among them, there were 48 SNPs with −log₁₀(P) values above 6, and 13 genes were annotated. Among these 48 SNPs, the SNP molecular marker significantly associated with the disease-resistant phenotype of sheep brucellosis was screened. Finally, a SNP molecular marker related to the resistance to brucellosis in sheep was obtained, where a polymorphic site of the SNP molecular marker was located at 35,704,228 bp on chromosome 3 of sheep (Oar_v1.0, November 2017). This site had a T/C base mutation, which was significantly correlated with the resistance to brucellosis in sheep. The cELISA value, iELISA value, and FPA value of sheep individuals with the “TT” homozygous genotype and the “CC” homozygous genotype of the polymorphic site were significantly lower than those of the “TC” heterozygous genotype (p<0.01). That is, compared with the “TC” heterozygous genotype, sheep with the “TT” homozygous genotype and the “CC” homozygous genotype showed stronger resistance to brucellosis, and “CC” and “TT” were the dominant genotypes of sheep resistant to Brucella. The SNP molecular marker corresponded to the sequence shown in SEQ ID NO: 1, where the polymorphic site was located at 101 bp, and the polymorphism was T or C.

Based on the SNP molecular marker, KASP primers for detecting the SNP molecular marker were further developed, as follows:
Primer X:
5′-gaaggtgaccaagttcatgctGGAGCCGGTGCCTCATGACT-3′ (SEQ ID NO: 5, where lowercase letter part represented a specific fluorescent tag sequence FAM);
Primer Y:
5′-gaaggtcggagtcaacggattGAGCCGGTGCCTCATGACC-3′ (SEQ ID NO: 6, where lowercase letter part represented a specific fluorescent tag sequence VIC);
Primer R: 5′-CAATGCTATCCAAAGCCTGGAGCTT-3′ (SEQ ID NO: 4).

The KASP primers could distinguish three genotypes, namely, the “TC” heterozygous genotype, the “TT” homozygous genotype, and the “CC” homozygous genotype, in the polymorphic site of the SNP molecular marker in the sheep population of this example.

### Example 2 Use of SNP Molecular Marker Associated with Resistance to Brucellosis in Sheep

The SNP molecular marker associated with resistance to brucellosis in sheep developed in Example 1 was validated in an expanded population, as follows:

1. Collection of Blood Samples from a Sheep Individual to be Tested and Identification of an Antibody Concentration in Serum
Jugular Blood was Collected from 129 Unvaccinated Suffolk Sheep, 126 East Friesian sheep, 129 White Suffolk sheep, and 27 Texel sheep from farms naturally infected with Brucella. iELISA was conducted to detect the antibody concentration of Brucella in serum. An iELISA value of an individual could be used as an indicator to characterize disease resistance, that is, a lower iELISA value indicated a stronger individual ability to resist brucellosis, and vice versa. In the example, a determination threshold for negative and positive iELISA results was set to 15.
2. Extraction of Genomic DNA from a Sheep Blood Sample to be Tested

A genomic DNA in the sheep blood sample to be tested was extracted by magnetic bead method.

### 3. Amplification of SNP Molecular Marker Fragment

The genomic DNA extracted in 2 was used as a template and the KASP primer combination (SEQ ID NO: 5 to SEQ ID NO: 6, SEQ ID NO: 4) developed in Example 1 was used for PCR amplification, as follows:

### (1) KASP Amplification System:

A 1.6 μL reaction system included: 0.8 μL of (50-100) ng/μL genomic DNA, 0.022 μL of primer mixture (preferred primer mixture ratio: 60 μL each of 100 μmol/μL forward primers Primer X and Primer Y, 150 μL of 100 μmol/μL reverse universal primer R, 230 μL of 10 mM Tris HCl), 0.4 μL of 2× Master mix, and adding double distilled water to make up to 1.6 μL.

The above reaction system was a preferred reaction system for the Douglas Array Tape platform. Other reasonable reaction systems could also achieve a same detection purpose.

The 2×Master mix included a fluorescent probe A, a fluorescent probe B, a quenching probe A, and a quenching probe B, as well as high-fidelity Taq enzyme, dNTP, and Mg²⁺. The fluorescent probe A had a sequence of 5′-GAAGGTGACCAAGTTCATGCT-3′ (SEQ ID NO: 7), with an FAM fluorophore linked to a 5′ terminus; the fluorescent probe B had a sequence of 5′-GAAGGTCGGAGTCAACGGATT-3′ (SEQ ID NO: 8), with a VIC fluorophore linked to a 5′ terminus; the quenching probe A had a sequence of 5′-AGCATGAACTTGGTCACCTTC-3′ (SEQ ID NO: 9), with a quenching group BHQ linked to a 3′ terminus; and the quenching probe B had a sequence of 5′-AATCCGTTGACTCCGACCTTC-3′ (SEQ ID NO: 10), with a quenching group BHQ linked to a 3′ terminus.

### (2) PCR Reaction Conditions:

DNA fragment amplification: initial denaturation at 94° C. for 15 min, one cycle; denaturation at 94° C. for 20 s, gradient annealing and extension at 61° C. to 55° C. for 60 s, 10 cycles, with 0.6° C. reduced in each cycle.

Fluorescence signal enhancement: denaturation at 94° C. for 20 s, annealing and extension at 55° C. for 60 s, 26 cycles.

4. The PCR Amplification Products were Detected Using the Douglas Array Tape Platform to Obtain the Genotype of the Polymorphic Site of the SNP Molecular Marker.

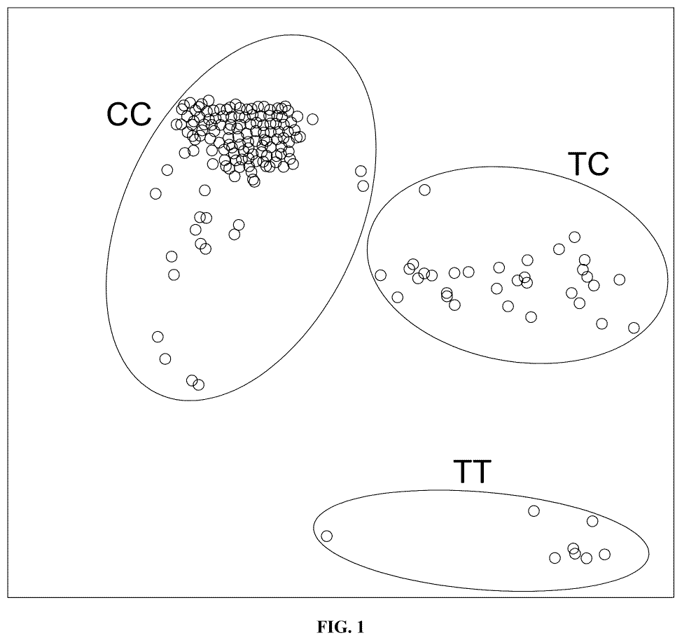

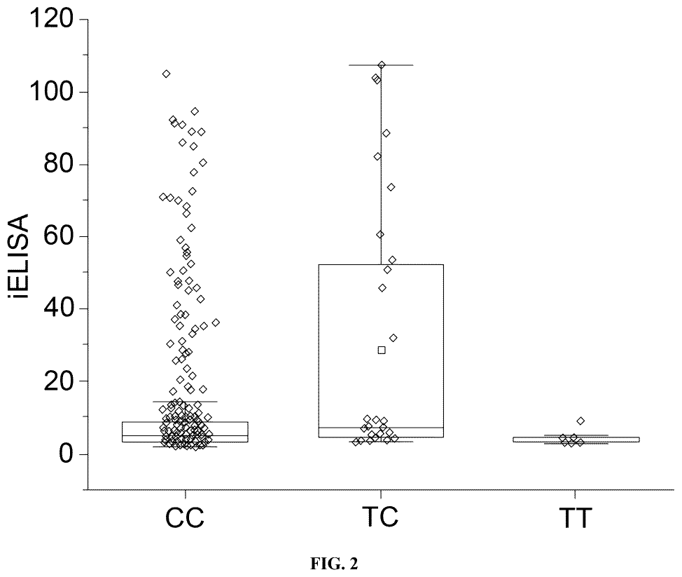

The genotype of the polymorphic site of the SNP molecular marker for each of the 411 sheep was tested. The results were shown in Table 1 and FIG. 1, and three genotypes of “CC”, “TC”, and “TT” were distinguished in the tested sheep population. The genotype was associated with the Brucella resistance trait, and the results were shown in FIG. 2. The iELISA values of sheep individual with the “CC” and “TT” genotypes were significantly lower than those with the “CT” genotype (p<0.01), indicating that the brucellosis resistance of sheep individual with the “CC” and “TT” genotypes was higher than that with the “CT” genotype. This also proved that “CC” and “TT” were the dominant genotypes of brucellosis-resistant sheep, and the SNP molecular marker provided by the present disclosure had a high accuracy in identifying Brucella resistance trait.

TABLE 1
Number of individuals with different genotypes at polymorphic
site of SNP molecular marker in sheep population

| | Genotype | | |
|---|---|---|---|
| | CC | TC | TT |
| Number of individuals | 371 | 32 | 8 |

Finally, it should be noted that the foregoing embodiments are only used to illustrate the technical solutions of the present disclosure, and are not intended to limit the present disclosure. Although the present disclosure is described in detail with reference to the foregoing embodiments, those of ordinary skill in the art should understand that they can still modify the technical solutions described in the foregoing embodiments, or make equivalent substitutions to some technical features therein. These modifications or substitutions do not make the essence of the corresponding technical solutions depart from the spirit and scope of the technical solutions in the embodiments of the present disclosure.

What is claimed is:

1. A kit, comprising a primer combination; wherein
the primer combination is used for amplifying a sheep single nucleotide polymorphism (SNP) molecular marker; the primer combination comprises primers having sequences shown in SEQ ID NO: 2 to SEQ ID NO: 4, wherein the primers having the sequences shown in SEQ ID NO: 2 to SEQ ID NO: 3 serve as forward primers, and the primer having the sequence shown in SEQ ID NO: 4 serves as a reverse universal primer; the sheep SNP molecular marker is associated with resistance to brucellosis in sheep, and has a nucleotide sequence shown in SEQ ID NO: 1 in which a polymorphism is T/C at 101^{st} base pair (bp); the sheep SNP molecular marker has the nucleotide sequence shown in SEQ ID NO: 1, a polymorphic site is located at 101^{st} bp of the nucleotide sequence shown in SEQ ID NO: 1, and the polymorphism is the T/C; the sheep SNP molecular marker is prepared by amplifying a sheep genome as a template using the primers having the sequences shown in SEQ ID NO: 2 to SEQ ID NO: 4; and a genotype being selected from a sheep population consisting of TT and CC corresponds to high resistance to brucellosis while the genotype being TC corresponds to low resistance to the brucellosis in the polymorphic site of the sheep SNP molecular marker.

2. A method of use of a sheep SNP molecular marker or a detection primer of the sheep SNP molecular marker in a treatment selected from any one of the following 1) to 8):
1) detecting or assisting in detecting a resistance level of sheep to brucellosis;
2) preparing a reagent for detecting or assisting in detecting the resistance level of the sheep to the brucellosis;
3) screening or identifying a sheep individual with high resistance to the brucellosis;
4) preparing a reagent for screening or identifying the sheep individual with the high resistance to the brucellosis;
5) conducting early prediction of resistance to brucellosis in sheep;
6) preparing a reagent for the early prediction of the resistance to brucellosis in sheep;
7) conducting molecular marker-assisted breeding for sheep with resistance to the brucellosis; and
8) conducting breed improvement for the sheep with the resistance to the brucellosis; wherein
the sheep SNP molecular marker has a polymorphic site located at position 35,704,228 of chromosome 3 of a sheep reference genome with the version of Oar_v1.0 on November 2017, and a polymorphism is T/C.

3. The method according to claim 2, wherein the sheep SNP molecular marker has a nucleotide sequence shown in SEQ ID NO: 1, the polymorphic site is located at 101^{st} bp of the nucleotide sequence shown in SEQ ID NO: 1, and the polymorphism is the T/C; and/or,
a detection primer of the sheep SNP molecular marker comprises primers having sequences shown in SEQ ID NO: 2 to SEQ ID NO: 4, wherein the primers having sequences shown in SEQ ID NO: 2 to SEQ ID NO: 3 serve as forward primers, and the primer having a sequence shown in SEQ ID NO: 4 serves as a reverse universal primer.

4. The method according to claim 2, wherein a genotype being selected from the group consisting of TT and CC corresponds to high resistance to the brucellosis while the genotype being TC corresponds to low resistance to the brucellosis in the polymorphic site of the sheep SNP molecular marker.

5. The method according to claim 3, wherein a genotype being selected from the group consisting of TT and CC corresponds to high resistance to the brucellosis while the genotype being TC corresponds to low resistance to the brucellosis in the polymorphic site of the sheep SNP molecular marker.
